(19) United States
(12) Patent Application Publication
(10) Pub. No.: US 2021/0361892 A1
(43) Pub. Date: Nov. 25, 2021

(54) Decontamination Device for Inhaled and Exhaled Ventilator Gases

(71) Applicant: Nader M. Habashi, Annapolis, MD (US)

(72) Inventor: Nader M. Habashi, Annapolis, MD (US)

(21) Appl. No.: 17/325,158

(22) Filed: May 19, 2021

Related U.S. Application Data
(60) Provisional application No. 63/027,342, filed on May 19, 2020.

(51) Int. Cl. A61M 16/00; A61L 9/20

(52) U.S. Cl. CPC A61M 16/009; A61M 2206/16; A61L 9/20

(57) ABSTRACT

The invention discloses a decontamination device for decontaminating inhaled and/or exhaled gases in a ventilator. The decontamination device comprises a gas inlet and outlet for inspiratory and expiratory gas flow, and a chamber for gas flow. The chamber is configured to connect the gas inlet and outlet. The chamber comprises at least one ultraviolet (UV) light source integrate inside the chamber that preferably emits in the UVC range, wherein the UV light source is preferably a UVC light-emitting diode. The UV light source is configured to produce UV light to decontaminate contaminants in the inhaled and/or exhaled gases by minimizing the distance between the contaminants in the gas flow and the light source, maintaining the gas flow in a gas flow path long enough to deactivate the contaminants, increasing the intensity of the light source to optimize decontamination, and increasing turbulence to redirect the gas flow and prevent boundary layers and shadowing.

### CROSS-REFERENCE TO RELATED APPLICATIONS AND CLAIM TO PRIORITY

This application relates to Provisional Application Ser. No. 63/027,342 filed May 19, 2020, the disclosure of which is incorporated herein by reference and to which priority is claimed.

### FIELD OF THE DISCLOSURE

The invention generally relates to a decontamination device as well as a ventilator comprising a decontamination device. More specifically, the invention relates to a device that uses ultraviolet (UV) light to decontaminate inhaled and/or exhaled gases from a ventilator and a ventilator comprising such a device.

### BACKGROUND OF THE DISCLOSURE

Currently, infectious diseases such as influenza, SARS, COVID-19, coronavirus, bacteria, and other viruses, cause mortality and morbidity via respiratory infection. Such infectious diseases affect respiratory system, including lungs of a patient and thus hampers the patient's ability to breathe and exchange oxygen and carbon dioxide. With the advancement in technology, several devices such as ventilators play an important role in the care of patients with compromised lung function and are thus used to assist the patient's lungs to exchange oxygen and carbon dioxide.

Typically, modern ventilators have filters in a mechanical ventilator circuit. Such filters can be placed at a gas intake and in an expiratory circuit, and thus helps in protecting the patient from any airborne contaminants that might be in the gas supply system or in the entrained ambient air, as well as protecting health care professional from pathogens exhaled into the environment by the patient. It can be noted that contamination of expired gas includes microbial pathogens, volatile organic compounds, nonstandard expired gases (e.g., anesthetic gases), and residual nebulized medications. In situations where a turbine may be used to entrain environmental air and gas, and the environment may be contaminated, inspired gas may entrain pathogens or chemicals into the ventilator. Filtration of supplied gas prevents bacterial and particulate contamination of an inspiratory limb of a ventilator. Typically, gases exhaled by the patient are discharged in the environment. Without filtration, expired pathogens are discharged into the environment and can mix with the air of the Intensive Care Unit (ICU) or an operating room. Therefore, the filtration of expired gas prevents contamination of the ambient atmosphere, protecting healthcare workers, other patients, and visitors.

Patients exhale volatile organic compounds and microparticles of water apart from water vapor and carbon dioxide that can be laden with pathogens. Currently, filters have a recommendation for filtering contaminants with a particle size of 1-3 μm. However, 80% of the contaminants from an intubated patient are usually about 0.3-1 μm in diameter, with around 2,500 particles per breath. Further, a main determinant of particle numbers is the positive end expiratory pressure (PEEP) variable (a higher PEEP reflects more exhaled particles), whereas tidal volume and mode of ventilation do not generally affect the number of particles. For example, a more severely ill patient with a respiratory infection corresponds to a higher bioburden and a higher contamination of exhaled gas. Further, exhaled breath particle (EBPs) concentrations in patients with pneumonia are about 237.67-1,208.13 particles/breath (0.2-2.196 particles/mL) vs 2.35-887.69 particles/breath (0.005-1.928 particles/mL) for patients without pneumonia. In addition, severely ill patients require more ventilator support and higher levels of PEEP. The median EBP concentration from mechanically ventilated patients with high PEEP level (5 cmH₂O) is 77.08 particles/breath (0.184 particles/mL), which significantly exceeds that for patients with low PEEP level <5 cmH₂O 13.92 particles/breath (0.027 particles/mL), p=0.003. PEEP levels in COVID 19 patients are generally >10 cmH₂O.

Thus, even with these filters, there is a serious risk of environmental contamination. The filters are not efficient in filtering out the contaminants in the inhaled or exhaled gases, and thus enter the patient's lungs and the patient's ambient environment, which subjects the patient and those exposed to the environment around the patient to a risk of respiratory infections from contaminants. This risk is due not only to the difficulty in filtering contaminants in the inhaled or exhaled gases, but also to the high clinical prevalence of respiratory infection causing contaminants. For example, during SARS, the ICU room contamination was high despite the use of expiratory filters. Contaminated condensates can potentially pass through some filters under typical pressures encountered during mechanical ventilation. Without filtration, expired pathogens tend to mix in the air of the ICU, the operating room, or elsewhere such as during transportation of patients within a hospital for treatment or to another hospital (inter or intra hospital transport).

Filtration of expired gas is currently used to minimize environmental contamination from patients who have respiratory illnesses such as COVID 19. In addition, masks are used, but the masks are prone to leakages. Further, the efficacy of expiratory filters is unknown in testing and raises the concern for risks of environmental contamination such as in the intensive care unit (ICU), operating room (OR) or during inter or intra-hospital transport. Current filters undergo in vitro testing based on estimates of particle size blockage. However, clinically relevant variables influence the efficacy of filtration including those that are encountered clinically such as gas cooling and condensates of water vapor and positive pressure in the expiratory circuit, which increases expiratory resistance.

To overcome some of the above-mentioned drawbacks of contaminants in the inhaled or exhaled gas, ultraviolet (UV) light is used to decontaminate gas flow. However, UV light used to decontaminate the contaminants in the inhaled or exhaled air are currently not efficient. Therefore, there is a need for an improved device for protecting the patient and reducing the risk of exposure to infectious micro-aerosols from patients receiving ventilation.

### SUMMARY OF THE DISCLOSURE

It is an object of the invention to provide a decontamination device that does not interfere with patient's breathing by reducing ventilator flow which increases resistance.

It is another object of the invention to provide a decontamination device wherein the gas flow in the device is passive such as not to interfere with the native gas flow from the ventilator, and which preferably does not contain valves and does not change the gas flow.

It is another object of the invention to provide a decontamination device that can be used on inspiratory and/or expiratory side of a ventilator, but preferably on the expiratory side to prevent contamination of the environment from exhaled gas.

It is another object of the invention to provide a decontamination device that decontaminates a gas flow in a ventilator by creating turbulence to redirect gas flow, providing narrow gas pathways to maximize exposure surface to a UV light, increasing gas pathway length to maximize exposure time to a UV light, and varying the intensity of the UV light as needed to decontaminate the gas flow to 99.9%.

It is another object of the invention to provide a decontamination device with a configuration that prevents boundary layers and shadowing with narrow pathways, turbulence to redirect flow, vibrations, oscillations, or a combination thereof.

It is another object of the invention to provide a decontamination device to optimize exposure time of the gas flow to the UV light relative to the intensity of the UV light.

It is another object of the invention to provide a decontamination device that decontaminates gas flow from a ventilator with a UVC light source, preferably a light-emitting diode UVC light or other safe and non-heat producing UVC light source.

It is another object of the invention to provide a decontamination device for invasive use with a ventilator, which has no possibility of leakage as with mask. The gas flow for intubated patients is contained because all gas flows through the ventilator when a patient is intubated.

It is another object of the invention to provide a decontamination device that can be retrofitted into an existing ventilator or built into a ventilator during ventilator manufacturing.

It is another object of the invention to provide a decontamination device for decontaminating inhaled and/or exhaled gases, in association with a ventilator. The decontamination device comprises a gas inlet and a gas outlet for inspiratory and expiratory gas flow. The decontamination device further comprises a chamber for gas flow. The chamber is configured to connect the gas inlet to the gas outlet. Further, the chamber comprises at least one ultraviolet (UV) light source integrated inside the chamber. The UV light source is configured to produce UV light to decontaminate contaminants in the inhaled and/or exhaled gases by minimizing the distance between the contaminants in the gas flow and the UV light source (i.e., expose the contaminants to the UV light), by maintaining the gas flow in a gas flow path long enough to deactivate the contaminants within 6 seconds, preferably within 3 seconds, even more preferably within ½ second in the case of a high respiratory frequency, and by increasing turbulence to prevent boundary layers and shadowing. In addition, the gas flow is passive to minimize resistance and to have the least impact on the native flow/exhaust of the ventilator. According to another embodiment, the decontamination device is integrated with the gas flow path of the native device, and the decontamination chamber exposes all of the gas inflow and out flow to decontamination.

In one embodiment, the turbulence is created by at least one element integrated within the chamber to redirect gas flow and prevent the formation of a boundary layers or shadowing of the UV light source. Further, the at least one element may be a baffle, a flow plate, or some other structure that would be known to one of skill in the art to redirect the gas flow and create turbulence. The flow plate is configured to disperse contaminants in the inhaled and/or exhaled gases. In one embodiment, the gas inlet and the gas outlet are configured to be connected to an inspiratory limb and/or an expiratory limb of the ventilator. Further, the chamber may comprise a reflective surface to amplify the UV light source penetration. In addition, the reflective surface may be made of aluminum or reflective copper. In another embodiment, the internal surfaces may be coated with antimicrobial coatings such as copper oxide or cold copper spray. In another embodiment, the chamber may be shielded by a casing to prevent exposure of the UV light to an external environment.

In another embodiment, the chamber comprises one or more gas flow paths for the flow of the inhaled and/or exhaled gases between the gas inlet and the gas outlet. Further, the one or more gas flow paths may comprise at least one of a spiral, coil, corkscrew, or other design that maximizes exposure of the inhaled and/or exhaled gases to the UV light. Such a design minimizes or eliminates boundary layers or shadowing effects to maximize pathogen exposure to the UV light.

The at least one UV light source is selected from the group comprising of UV lamps, ultraviolet-light-emitting diodes (UV-LEDs), and other preferably non-heat producing sources of UV light. Further, the at least one UV light source is at least one UV light tube supported by one or more support plates and end supports, through which the gas flows. Further, the UV light comprises a wavelength in a range of about 200-280 nm, more preferably about 254 nm or far UVC. Further, an effective area of decontamination is based at least on the length and diameter of the gas path, location and quantity of the UV lights, and intensity of the UV light. The intensity of the UV light is based at least on the gas path. Further, the distance between the contaminants in the gas flow and the light source is minimized to expose the contaminants to the UV light. Additionally, although there is inherent turbulence in ventilator gas flow, turbulence can be increased by adding a baffle or a vibration/oscillation to minimize or eliminate boundary layers or shadowing and thereby maximize pathogen exposure to the UV light.

Such decontamination devices provide efficient decontamination of contaminants from the inhaled or exhaled gases, in association with a ventilator. In one embodiment, the contaminants comprise at least bacterial, viral, COVID 19, or other pathogens.

The invention depends on the cyclic nature of ventilation in which there is an inherent time lag between cycles of inspiration and expiration. The decontamination system allows the entire gas output to flow through the chamber (as opposed to just a fraction of the total flow) resulting in the entirety of the inhaled or exhaled gas to be decontaminated and eliminating the need for recirculation. It is another object of the invention not to rely on filter technology to eliminate environmental contamination.

According to a preferred embodiment, the decontamination device of the invention does not interfere with the patient's breathing by reducing expiratory flow, increasing resistance (such as by using filters or filtration methods) and does not interfere or divert the native gas flow from the patient to the ventilator. Preferably, the decontamination device does not contain valves or redirect flow to add resistance.

Another aspect of the invention comprises a ventilator comprising a decontamination device as described herein, as well as a method of decontaminating a ventilator gas flow by installing a decontamination device as described herein into the gas flow of a ventilator.

These and other examples of the invention will be described in further detail below.

### BRIEF DESCRIPTION OF THE DRAWINGS

The accompanying drawings illustrate various embodiments of systems, methods, and embodiments of various aspects of the disclosure. A person of ordinary skill in the art will appreciate that the illustrated element boundaries (e.g., boxes, groups of boxes, or other shapes) in the figures represent one example of the various boundaries representative of the disclosed invention. It may be that in some examples one element may be designed as multiple elements or that multiple elements may be designed as one element. In other examples, an element shown as an internal component of one element may be implemented as an external component in another, and vice versa. Furthermore, elements may not be drawn to scale. Non-limiting and non-exhaustive descriptions of the disclosure are described with reference to the following drawings. The components in the figures are not necessarily to scale, emphasis instead being placed upon the illustrated principles.

Various embodiments will hereinafter be described in accordance with the appended drawings, which are provided to illustrate but not to limit the scope of the disclosure in any manner, wherein similar designations denote similar elements, and in which:

FIG. 1 illustrates a schematic diagram of a decontamination device for decontaminating inhaled and/or exhaled gases, according to a first embodiment of the disclosure;

FIG. 2 illustrates a schematic diagram of a decontamination device for decontaminating inhaled and/or exhaled gases, according to a second embodiment of the disclosure;

FIGS. 3A and 3B illustrate a schematic diagram of decontamination device for decontaminating inhaled and/or exhaled gases, according to a third embodiment of the disclosure;

FIG. 4 illustrates a schematic diagram of a decontamination device for decontaminating inhaled and/or exhaled gases, according to a fourth embodiment of the disclosure;

FIG. 5 illustrates a schematic diagram of a system of decontaminating air entrained into a ventilation system, according to an embodiment of the disclosure;

FIG. 6 illustrates a schematic diagram of a system having a decontamination device inside a ventilation system, according to an embodiment of the disclosure;

FIG. 7 illustrates a schematic diagram of a decontamination device coupled to an inspiratory limb of a ventilation system, according to an embodiment of the disclosure; and

FIG. 8 illustrates a schematic diagram of a decontamination device coupled to an expiratory limb of a ventilation system, according to an embodiment of the disclosure.

### DETAILED DESCRIPTION

Reference will now be made in detail to specific embodiments or features, examples of which are illustrated in the accompanying drawings. Wherever possible, corresponding, or similar reference numbers will be used throughout the drawings to refer to the same or corresponding parts. Moreover, references to various elements described herein, are made collectively or individually when there may be more than one element of the same type. However, such references are merely exemplary in nature. It may be noted that any reference to elements in the singular may also be construed to relate to the plural and vice-versa without limiting the scope of the disclosure to the exact number or type of such elements unless set forth explicitly in the appended claims.

In the interest of clarity, not all of the routine features of the implementations described herein are shown and described. It will, of course, be understood that in the development of any such actual implementation, numerous implementation-specific decisions may be made in order to achieve the specific goals, such as compliance with manufacturing and business-related constraints, and that these specific goals will vary from one implementation to another and from one manufacturer to another. Moreover, it will be understood that such a manufacturing effort might be complex and time-consuming, but would nevertheless be a routine undertaking of engineering for those of ordinary skills in the art having the benefit of this disclosure.

Some embodiments of this disclosure, illustrating all its features, will now be discussed in detail. The words “comprising,” “having,” “containing,” and “including,” and other forms thereof, are intended to be equivalent in meaning and be open-ended in that an item or items following any one of these words is not meant to be an exhaustive listing of such item or items, or meant to be limited to only the listed item or items.

It must also be noted that as used herein and in the appended claims, the singular forms “a,” “an,” and “the” include plural references unless the context dictates otherwise. Although any number of systems and methods similar or equivalent to those described herein can be used in the practice or testing of embodiments of the disclosure, the preferred systems, and methods are now described.

Embodiments of the disclosure will be described more fully hereinafter with reference to the accompanying drawings in which like numerals represent like elements throughout the several figures, and in which example embodiments are shown. Embodiments of the disclosure may, however, be embodied in alternative forms and should not be construed as being limited to the embodiments set forth herein. The examples set forth herein are non-limiting examples and are merely examples among other possible examples.

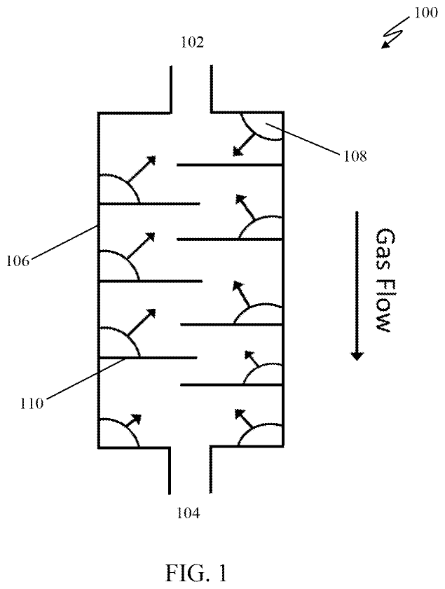

FIG. 1 illustrates a schematic diagram of a decontamination device 100 for decontaminating inhaled and/or exhaled gases, according to a first embodiment of the disclosure. The decontamination device 100 may comprise a gas inlet 102 and a gas outlet 104. In one embodiment, the gas inlet 102 may be connected to an inspiratory limb and/or an expiratory limb of a ventilator. In an alternate embodiment, the gas outlet 104 may be connected to an inspiratory limb and/or an expiratory limb of the ventilator.

Further, the decontamination device 100 may comprise a chamber 106, for gas flow. The chamber 106 may be configured to connect the gas inlet 102 to the gas outlet 104. The chamber 106 may comprise at least one Ultraviolet (UV) light source 108 integrated inside the chamber 106. In another embodiment, the at least one UV light source 108 may be integrated at one or more corners of the chamber 106, to expose the gas flow, between the gas inlet 102 and the gas outlet 104, to the UV light. Further, the at least one UV light source 108 may be configured to produce UV light to decontaminate contaminants in the inhaled and/or exhaled gases by minimizing the distance between the contaminants in the gas flow and the UV light source, by maintaining the gas flow in a gas flow path long enough to deactivate the contaminants within 3-6 seconds, and by creating turbulence to prevent boundary layers and shadowing. According to a preferred embodiment, the distance between the contaminants in the gas flow and the UV light source is no more than 10 mm, preferably no more than 5 mm.

UV light is a type of electromagnetic radiation with wavelengths ranging from 10 nm to 400 nm. The wavelengths of the UV light are shorter than wavelengths of visible light. Further, the wavelengths ranging from 100 to 400 nm of ultraviolet radiation (UV light) are subcategorized into three different ranges: Ultraviolet C (UVC) ranging from 100-280 nm, Ultraviolet B (UVB) ranging from 280-315 nm, and Ultraviolet A (UVA) ranging from 315-400 nm. Further, UVA is capable of penetrating the skin and is responsible for up to 80% of skin ageing, from wrinkles to age spots. UVB can damage the DNA in the skin, leading to sunburn and eventually skin cancer. UVC consists of a shorter, more energetic wavelength of light and is particularly good at destroying genetic material, including in viral particles. The UVC light can penetrate the thin wall of a microscopic organism and destroy its nucleic acids.

Germicidal UV light, typically around 254 nm, is effective may adversely affect skin and eyes. According to another embodiment, the UV lights of the invention are encased in a chamber or internal to a ventilator to prevent environmental exposure. In another embodiment, far UVC may be used as the light source. Far-UVC light (207 to 222 nm) has been shown to be as efficient as conventional germicidal UV light in killing microorganisms and may be safer because it does not penetrate the skin and eyes.

Further, the UV light source may be any man-made ultraviolet light source including UV lamps, arc welding, mercury vapor lamps, UV LED lights, or preferably another non-heat producing UV light source. According to a preferred embodiment, UV LED lights may be used as the light source. UV LED lights may have a small size that facilitates incorporation into the decontamination device. Additionally, UV LEDs do not contain mercury, which alleviated the risk of human and environmental toxicity. Additionally, the UV LED intensity is not influenced by temperature change and no warm-up time is required for maximum intensity output. In addition, UVC LEDs have been shown to have much higher inactivating efficacy against bacteria, viruses, and fungi.

Thus, a UVC light may be used to decontaminate contaminants in the gas flow. In one embodiment, the contaminants may be selected from a group comprising of bacterial, viral, COVID 19, or other pathogens, without departing from the scope of the disclosure. It can be noted that the gas flow may correspond to the inhaled gases or the exhaled gases of a patient.

The chamber 106 may comprise a reflective surface to amplify the UV light source penetration. Further, the reflective surface may be made of aluminum or reflective copper. Further, an internal surface of the chamber 106 may be coated with antimicrobial coatings such as copper oxide or cold copper spray. As shown in FIG. 1, the chamber 106 may include at least one element 110 integrated within the chamber 106 to prevent formation of a boundary layer or shadowing of the contaminants from at least one UV light source 108 by creating a narrow pathway or turbulence. In another embodiment, the at least one element 110 may be lined within the chamber 106. Further, the at least one element 110 may ensure that the contaminants in the gas flow is exposed to the UV light. In another embodiment, the at least one element 110 may be at least one baffle fixed to the walls of the chamber 106.

According to the invention, the gas flow may be passive to minimize resistance and reducing the impact on the native flow of the ventilator. There are inherent risks when interfering with expiration of a ventilated patient. Unlike inspiration, which is a result of pressurized gas driven into the patient by the ventilator, expiration is passive and is a result of the thoracic recoil force and downstream resistance. Any device added to the expiratory side of a ventilator circuit must consider resistance and minimize it.

The decontamination device 100 prevents interference with the patient's breathing, by not altering the expiratory flow from the ventilator. Further, the at least one element 110 may create turbulence for increased exposure to optimize intensity and duration of UV light exposure. Further, the turbulence may help in disinfecting and circulating gas flow to prevent formation of a boundary layer or shadowing. The formation of a boundary layer may be eliminated by decreasing the distance between the at least one UV light source 108 and the contaminants and increasing the intensity of the UV lights. Further, the turbulence can further decrease the boundary layer and reduce shadowing to improve decontamination. However, turbulence may cause resistance to the gas flow, which can be offset with an increase in the functional area. Further, the gas path in the decontamination device 100 should be large enough to expose all gas flow to maximum UV light and to minimize resistance, and therefore not impact the patient's expiratory gas flow. In one embodiment, the gas flow in the gas flow path may be maintained long enough to deactivate the contaminants within 6 seconds.

In another embodiment, the at least one element 110 may be a flow plate that may be configured to disperse contaminants in the inhaled and/or exhaled gases. In one embodiment, the configuration of the gas flow path through the decontamination chamber, has a gas path distance from the light source of 22 mm or less allowing multidirectional gas exposure. In another embodiment, the chamber 106 of the decontamination device 100 may be square, rectangle or cylindrical in shape, preferably 4″ wide by 12″ deep, and the gas path may flow through a 22 mm tubing.

In another embodiment, the gas path may be impregnated with copper oxide. The use of copper oxide may produce potent anti-viral properties without altering their physical barrier properties. The use of an anti-viral gas path may significantly reduce the risk of contamination. It will be apparent to one skilled in the art that the above-mentioned shape of the chamber 106 has been provided only for illustration purposes, without departing from the scope of the disclosure. Such decontamination device 100 eliminates the need for filter technology to eliminate environmental contaminants or to filter exhaled gases. It can be noted that the decontamination device 100 may be retrofitted or may be built-into ventilators. Further, the decontamination device 100 eliminates the need for valves along the expiratory flow, which would affect a patient's breathing. In another embodiment, the decontamination device 100 may be configured to vibrate to increase movement of the contaminants and expose the gas flow to the UV light. For example, creating a low-level frequency with a speaker or a membrane that moves such as in an oscillating ventilator.

In another embodiment, the UV dose from the UV light source 108 may be defined as:

UV Dose=UV Intensity (mJ/cm²)×Exposure Time (seconds)

Further, based on data, viruses such as COVID 19 are sensitive to UVC and Far UVC (207 to 222 nm). Further to reach 99.9 the dose of 1.2-17 mJ/cm², the following data has been recorded:

| | k | D₉₀ | D₉₉ | D_{99.9} |
|---|---|---|---|---|
| Species | (cm²/mJ) | (mJ/cm²) | (mJ/cm²) | (mJ/cm²) |
| HCoV-229E | 4.1 | 0.56 | 1.1 | 1.7 |
| HCoV-OC43 | 5.9 | 0.39 | 0.78 | 1.2 |
| Influenza A | 1.8 | 1.3 | 2.6 | 3.8 |
| (H1N1)*^{†} | | | | |

It can be noted that the above data is documenting the relatively low dose needed to kill COVID 19 at a 20 cm distance for the UVC light source to the viral culture. This dose can be increased to achieve higher ultraviolet germicidal irradiation (UVGI) effect. According to a preferred embodiment, the intensity of the UV light is between about 0.010-110 mJ/cm², preferably between about 1.2-17 mJ/cm². Alternatively, the proximity to the UVC light source can be minimized. Further, the duration of exposure is regulated because the exposure time of the gas flow is relative to the intensity of the UV light. In another embodiment, the reduced distance between the UV light source and the gas flow may allow lower dosing to be applied as the intensity from the proximity to the light source is increased. Further, the chamber 106 may be equipped with a reflective surface to amplify the UV light source 108 and to reduce the physical number of UV light sources 108 needed to maintain optimize intensity.

In another embodiment, for a 30 cm distance for the UVC light source to the viral culture, the recorded data is shown (in table 1):

TABLE 1
UV-C lethal doses for SARS-CoV-2.

| Viral | UV-C dose | Exposure |
|---|---|---|
| inactivation (%) | (mJ/cm²) | time (s) |
| 90 | 0.016 | 0.01 |
| 99 | 0.706 | 0.32 |
| 99.9 | 6.556 | 2.98 |
| 99.99 | 31.880 | 14.49 |
| 99.999 | 108.714 | 49.42 |

In another embodiment, the gas-light exposure of the UVGI may be designed to achieve log 3 or greater reduction in pathogens. In order to achieve a log 3 level of reduction, the configuration of the decontamination device 100 may optimize variables including the UVC dose, the distance from the light source, duration of exposure as it related to distance of the gas path, and elimination of a boundary or shadow layer, UVC wavelength, and UVC light-emitting diodes (LEDs).

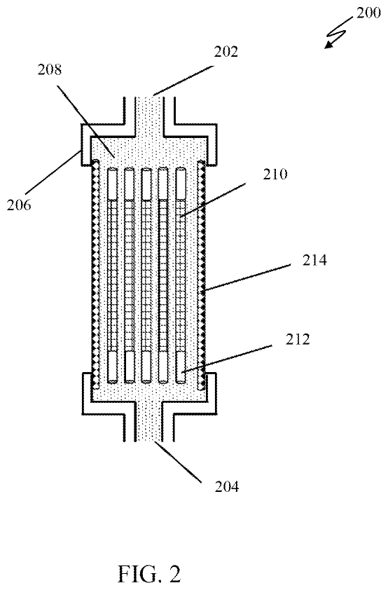

FIG. 2 illustrates a schematic diagram of a decontamination device 200 for decontaminating inhaled and/or exhaled gases, according to another embodiment of the disclosure. The decontamination device 200 may comprise a gas inlet 202 and a gas outlet 204. In one embodiment, the gas inlet 202 may be connected to an inspiratory limb and/or an expiratory limb of a ventilator. In an alternate embodiment, the gas outlet 204 may be connected to an inspiratory limb and/or an expiratory limb of the ventilator.

Further, the decontamination device 200 may comprise a chamber 206 for gas flow. It can be noted that the gas flow is from the gas inlet 202 towards the gas outlet 204. The chamber 206 may be configured to connect the gas inlet 202 to the gas outlet 204. The chamber 206 may comprise at least one gas flow path 208, and at least one UV light source 210 associated with each of the at least one gas flow paths 208. Further, the at least one UV light source 210 may emit radiation through the at least one gas flow path 208 to decontaminate the gas flow. Further, the at least one UV light source 210 may be supported in the decontamination device 200 by one or more supports 212. In another embodiment, the at least one UV light source 210 of the decontamination device 200, may be shielded from the exterior environment by a casing 214 to prevent the UV light from leaking outside the decontamination device 200. In an additional embodiment, the at least one gas flow tube 208 may have a coil or a corkscrew design (not shown) to increase exposure to the UV light. Further, use of the decontamination device 200 on an expiratory side of the ventilator may prevent contamination of the environment and the risk to healthcare providers and other patients around a ventilated patient.

It will be apparent to one skilled in the art that the above-mentioned components of the decontamination device 200 have been provided only for illustration purposes, without departing from the scope of the disclosure.

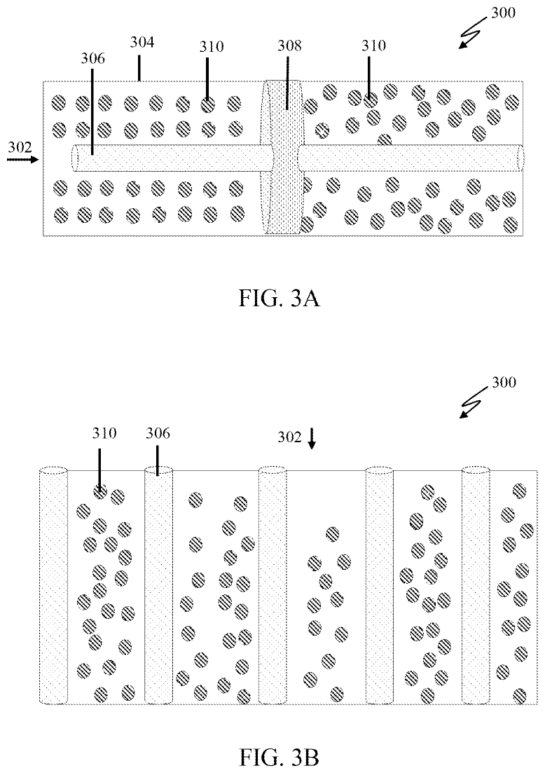

FIGS. 3A and 3B illustrate a schematic diagram of a decontamination device 300, according to yet another embodiment of the disclosure. The decontamination device 300 may allow gas flow 302 through the device 300. Further, the decontamination device 300 may comprise a chamber 304. The chamber 304 may comprise at least one UV light source 306 and at least one gas flow plate 308 to disperse contaminants 310 to expose the contaminants 310 to the at least one UV light source 306. In one embodiment, the contaminants 310 may be bacterial, viral, COVID 19, or other pathogens.

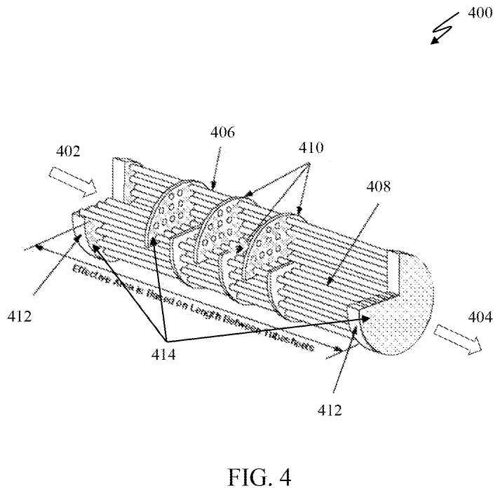

FIG. 4 illustrates a schematic diagram of a decontamination device 400, according to yet another embodiment of the disclosure. The decontamination device 400 may comprise a gas inlet 402 and a gas outlet 404. In one embodiment, the gas inlet 402 may be for an inspiratory or expiratory gas flow and the gas outlet 404 may be for an inspiratory or expiratory gas flow. Further, the decontamination device 400 may also comprise a chamber 406, for gas flow. It can be noted that the gas flow is from the gas inlet 402 towards the gas outlet 404.

Further, the chamber 406 may be coupled to the gas inlet 402 and the gas outlet 404. The chamber 406 may comprise at least one UV light tube 408. Further, the gas flow may be along the at least one UV light tube 408. Further, the at least one UV light tube 408 may be supported by one or more support plates 410 and a pair of end supports 412. Further, the one or more support plates 410 and the pair of end supports 412 may include through holes 414, for the gas flow and to disperse contaminants to increase decontamination. Further, the effective area of decontamination of the gas flow may be based on the length and diameter of the chamber and the quantity of the at least one UV light tubes 408 in the gas path. Further, the effective area of decontamination necessary may depend on the intensity of UV light in the at least one UV light tube 408. Additionally, the intensity of the UV light may be more or less depending on length of the gas path as well as the distance of the contaminants to the UV light tube 408. Further, the cross-sectional area to be decontaminated preferably allows UV penetration for complete sterilization of the gas flow. Therefore, the shorter the gas path length, the more the intensity of the UV light is required. It should be noted that the gas path needs to be long enough to allow enough contact time for the gas flow to be decontaminated within about 6 seconds, preferably about 3 seconds. In one embodiment, the contact time and exposure to the UV light may be increased by feeding the gas flow through a coil or spiral pathway. Alternatively, the gas path may flow through multiple smaller compartments to increase exposure to the UV light source.

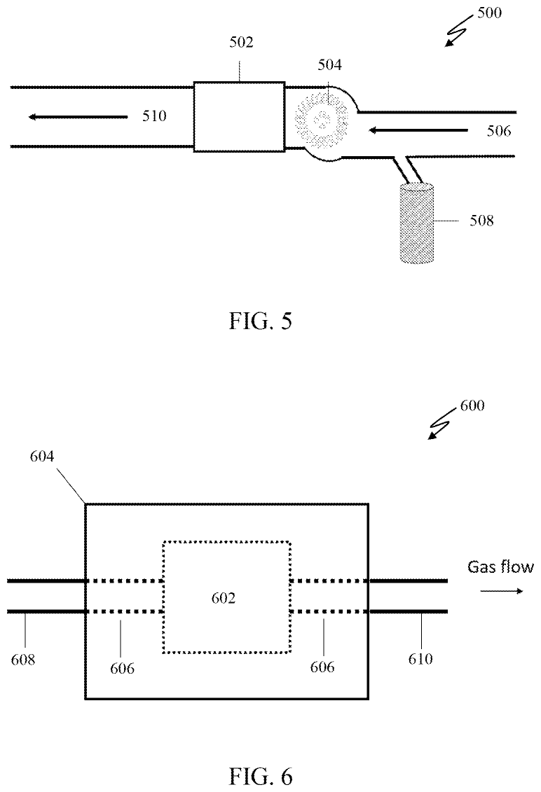

FIG. 5 illustrates a system 500 for decontaminating air that may be contaminated in an open environment. The system 500 is used for decontaminating air entrained into a ventilation system, according to an embodiment of the disclosure. As seen in FIG. 5, a decontamination device 502 may be retrofitted to an inspiratory limb of a ventilation system, for example, downstream of a turbine 504. Further, air 506 may be entrained by the turbine 504 and mixed with oxygen from an oxygen supply 508, at a desired ratio to create a gas flow at a certain concentration. Further, a filter may also be added to the inspiratory side to remove water and larger particles/contaminants. The gas flow may then be fed into the decontamination device 502, and decontaminated with a UV light source according to any of the above embodiments. Once the gas flow 510 is decontaminated, it is fed to the ventilation system to ventilate a patient. Further, the decontamination device 502 eliminates the possibility of leakage of gas, as the gas is fed to a patient through the ventilator by intubation. It can be noted that the decontamination device 502 may be a decontamination device as shown in any of FIG. 1, 2, 3A, 3B, or 4.

FIG. 6 illustrates a schematic diagram of a system 600 having a decontamination device 602 inside a ventilator apparatus 604, according to another embodiment of the disclosure. As seen in FIG. 6, the decontamination device 602 may be installed inside the ventilator apparatus 604 in the gas path 606. The system may include a gas inlet 608. The gas inlet 608 may be a high-pressure connecter for the gas source coming into the ventilator apparatus 604. Further, the system may include an internal gas path 606 inside the ventilator apparatus 604. Further, the system may include an inspiratory connecter 610. The inspiratory connector 610 may come out of the ventilator apparatus 604 such that the inspiratory limb of the ventilator apparatus 604 may be connected to the patient. It can be noted that the decontamination device 602 may be a decontamination device as shown in any of FIG. 1, 2, 3A, 3B, or 4.

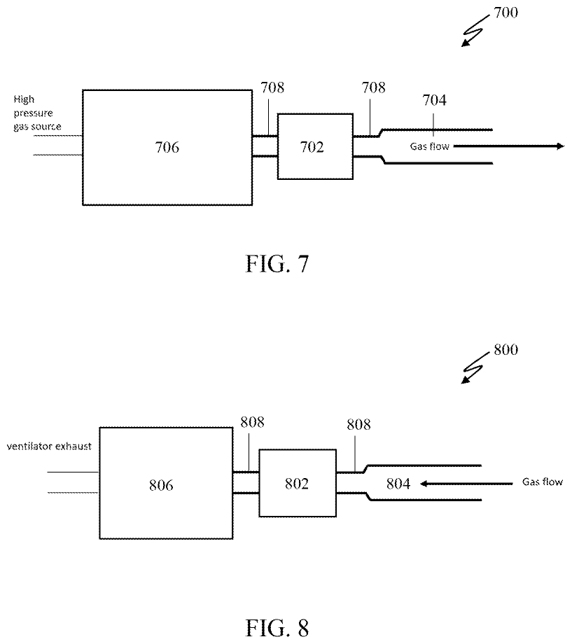

FIG. 7 illustrates a schematic diagram of a system 700 having a decontamination device 702 coupled to an inspiratory limb 704 of a ventilation system 706, according to an embodiment of the disclosure. The decontamination device 702 may be retrofitted to the inspiratory limb 704 of the ventilation system 706. Further, the system 700 may include a connector 708 for connecting the decontamination device 702 to the ventilation system's inspiratory connecter port and the ventilator circuit. Further, the gas flow may then be fed into the decontamination device 702, and decontaminated with the UV light according to any of the above embodiments. In one embodiment, a high-pressure gas source may be coupled to the ventilation system 706. It can be noted that the decontamination device 702 may be a decontamination device as shown in any of FIG. 1, 2, 3A, 3B, or 4.

FIG. 8 illustrates a schematic diagram of a system 800 having a decontamination device 802 coupled to an expiratory limb 804 of a ventilation system 806, according to an embodiment of the disclosure. The decontamination device 802 may be retrofitted to the expiratory limb 804 of the ventilation system 806. Further, the system 800 may include a connector 808 for connecting the decontamination device 802 to the ventilation system's the expiratory connecter port and the ventilator circuit. Further, the gas flow may then be fed into the decontamination device 802, and decontaminated with UV light according to any of the above embodiments. In one embodiment, a ventilator exhaust may be coupled to the ventilation system 806. It can be noted that the decontamination device 802 may be a decontamination device as shown in any of FIG. 1, 2, 3A, 3B, or 4.

Modern ventilators are highly specialized, and their behavior may be altered, or they may fail (sound an alarm) if expiratory gas flow is altered, or resistance is increase putting the patient at risk. During normal operation of a ventilator, an operator may choose to set a range of frequencies that are dictated clinically. Thus, the exposure time may vary significantly. As a result, countermeasures are needed to deal with the potential for less UVC exposure time without slowing or diverting the expiratory gas flow and affecting the patient's expiratory gas flow. Therefore, according to the invention, it is important to maximize the UVC intensity with the least amount of heat (such as with an LED UVC), to minimize the distance to the UVC source by reducing the gas path, to increase the UVC contact length, to eliminate boundary layers and shadowing, to use biocidal surfaces such as copper, and to use the oscillation or vibration of gas to decontaminate the expiratory gas flow without affecting the expiratory gas flow rate from the patient.

While there is shown and described herein certain specific structures illustrating various embodiments of the invention, it will be manifest to those skilled in the art that various modifications and rearrangements of the parts may be made without departing from the spirit and scope of the underlying inventive concept and that the same is not limited to the particular forms herein shown and described except insofar as indicated by the scope of the appended claims.

What is claimed is:

1. A decontamination device for decontaminating inhaled and/or exhaled gases, in association with a ventilator, the decontamination device comprising:
a gas inlet and a gas outlet for inspiratory and/or expiratory gas flow; and
a chamber for gas flow, configured to connect the gas inlet to the gas outlet, the chamber comprising at least one ultraviolet (UV) light source integrated inside the chamber configured to produce UV light to decontaminate contaminants in the inhaled and/or exhaled gases by minimizing the distance between the contaminants in the gas flow and the UV light source, maintaining the gas flow in a gas flow path long enough to deactivate the contaminants, and optimizing the intensity of the UV light to deactivate the contaminants, and
wherein the gas flow is passive to minimize resistance.

2. The decontamination device as claimed in claim 1, wherein the distance between the contaminants in the gas flow and the UV light source is no more than 10 mm, preferably no more than 5 mm.

3. The decontamination device as claimed in claim 1, wherein the gas flow is maintained in the gas flow path long enough to deactivate the contaminants within 6 seconds, preferably within 3 seconds.

4. The decontamination device as claimed in claim 1, wherein the intensity of the UV light is between about 0.010-110 mJ/cm², preferably between about 1.2-17 mJ/cm².

5. The decontamination device as claimed in claim 1, further comprising an element configured to increase turbulence within the chamber to redirect the gas flow to prevent formation of a boundary layer or shadowing.

6. The decontamination device as claimed in claim 5, wherein the at least one element is a baffle or a flow plate.

7. The decontamination device as claimed in claim 6, wherein the flow plate is configured to disperse contaminants in the gas flow.

8. The decontamination device as claimed in claim 1, wherein the chamber comprises one or more gas flow paths for the flow of the inhaled and/or exhaled gases between the gas inlet and the gas outlet.

9. The decontamination device as claimed in claim 8, wherein the one or more gas flow paths comprise at least one of a spiral, coil, or corkscrew design, to increase the exposure of the contaminants in the gas flow to the UV light.

10. The decontamination device as claimed in claim 1, wherein the chamber comprises a reflective surface to amplify the UV light penetration.

11. The decontamination device as claimed in claim 10, wherein the reflective surface comprises aluminum or reflective copper.

12. The decontamination device as claimed in claim 1, wherein the chamber comprises a casing to prevent exposure of the UV light to an external environment.

13. The decontamination device as claimed in claim 1, wherein the UV light comprises a wavelength in a range of 200-280 nm.

14. The decontamination device as claimed in claim 1, wherein the at least one UV light source is selected from the group comprising of UV lamps and ultraviolet-light-emitting diodes (UV-LEDs).

15. The decontamination device as claimed in claim 1, wherein the at least one UV light source is at least one UV light tube supported by one or more support plates and end supports, along which the gas flows.

16. The decontamination device as claimed in claim 1, wherein an effective area of decontamination is based at least on the length and diameter of the gas flow path.

17. The decontamination device as claimed in claim 16, wherein the intensity of the UV light is based on the effective area of decontamination.

18. The decontamination device as claimed in claim 1, wherein the decontamination device is configured to vibrate to increase movement of gas molecules and expose the gas flow to the UV light.

19. A ventilator comprising the decontamination device as claimed in claim 1, wherein the gas inlet and the gas outlet are configured to be connected to an inspiratory limb and/or an expiratory limb of the ventilator.

20. A method of decontaminating gas flow from a ventilator, comprising installing the decontamination device as claimed in claim 1 in the inspiratory and/or expiratory gas flow of the ventilator and decontaminating the gas flow by reducing the contaminants, wherein the contaminants comprise at least one of bacterial, viral, COVID 19, or other pathogens.
